(19) United States
(12) Patent Application Publication
(10) Pub. No.: US 2007/0174985 A1
(43) Pub. Date: Aug. 2, 2007

(54) Toothbrush with tongue scraper/cleaner

(76) Inventor: Joe Steven JR., Wichita, KS (US)

Correspondence Address: Joe Steven, Jr. D.D.S., 232 N. Seneca, Wichita, KS, 67203, US

(21) Appl. No.: 11/245,982

(22) Filed: Jan. 27, 2006

(51) Int. Cl. A61B 17/24

(52) U.S. Cl. 15/111; 606/161

(57) ABSTRACT

A toothbrush comprising a handle at one end for which to hold the brush, and at the opposing end, a head containing bristles, provided for cleaning the teeth. On the opposite side of the bristles, on the same end of the toothbrush, are ridges evenly spaced, used for scraping the tongue to remove plaque, bacteria, dead tissue, food debris, etc. from the tongue area.

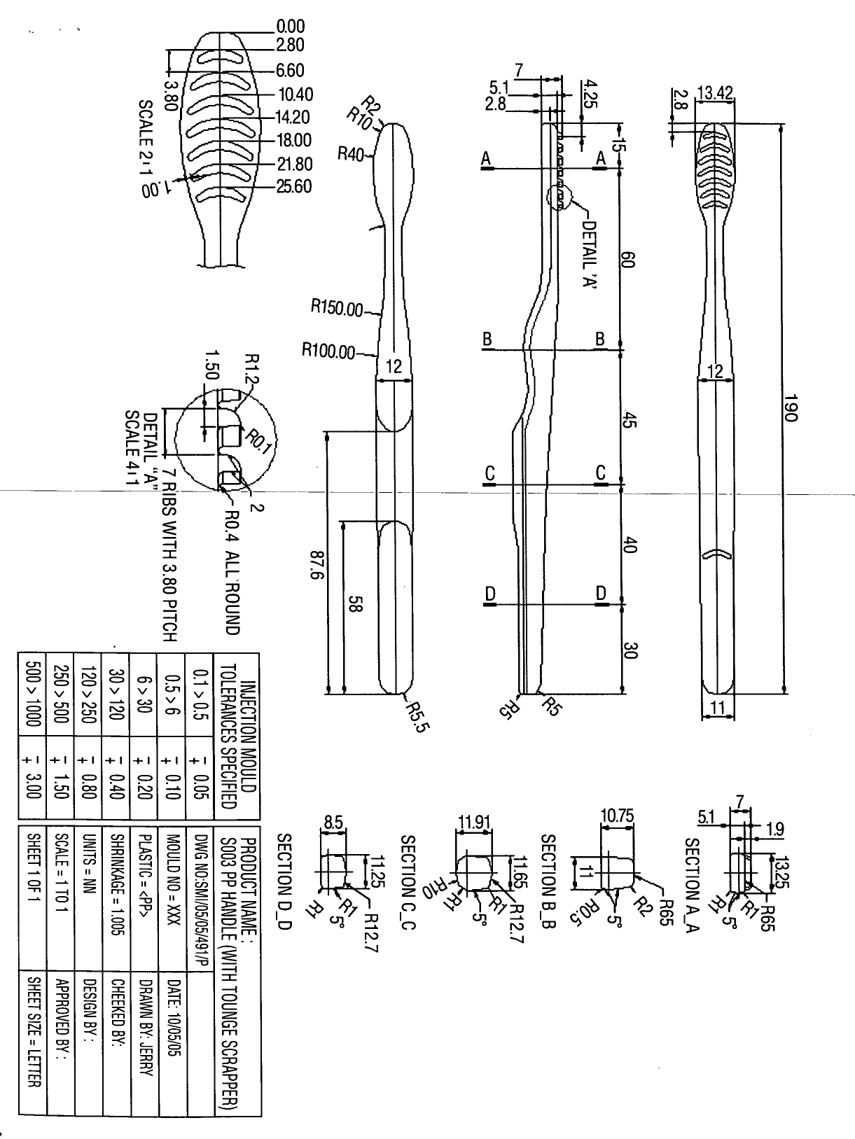

The KISCO Combo Toothbrush consists of a typical toothbrush with a handle at one end for which to hold the brush, and at the opposite end, a head containing bristles, provided for cleaning the teeth. On the opposite side of the bristles, on the same end of the toothbrush where the bristles are located, are ridges evenly spaced, used for scraping the tongue to remove plaque, bacteria, dead tissue, food debris, and etc. from the tongue area.

It looks very similar to a regular toothbrush except on the reverse side where the bristles are located (rotate the brush 180°) there are ridges processed into the acrylic designed for scraping and cleaning the tongue. It is designed small enough to be able to fit in the mouth and do a thorough job of brushing the teeth and also cleaning the tongue.

What is claimed is:

1. A toothbrush with tongue scraper/cleaner comprising:
a conventional toothbrush handle configured and adapted to be grasped by a human hand, the handle having a longitudinal axis, a neck emanating from said handle, a head region located distal to said neck, a tongue scraper located in said head region, said tongue brush including: a bristle base configured to be a substantially rigid platform that will support bristles and tongue scraper in use, said bristle base having a proximal end adjacent to said neck, a distal end, a top and a bottom, and a plurality of bristles emanating from said bristle base top, each of said bristles having a proximal end adjacent to said bristle base and a distal end, at least some of said bristles being tapered from their proximal end to their distal end to present a pointed bristle appearance, at least some of said bristles being of differing heights, said bristles being arranged generally in order of descending height from said bristle base distal end to said bristle base proximal end, said bristles and said bristle base being formed as a single unitary component from the same material via an injection molding process, said bristles emanating from said bristle base top in an individually-mounted and free-standing non-tufted configuration, and a tongue scraper emanating from said bristle base top, said tongue scraper including: a series of raised scraper blades having sufficient flexibility to be useful in removing undesirable material from a human tongue by a scraping action.

2. A device as recited in claim 1 wherein said handle, neck, bristle base, and tongue scraper are formed as a single unitary component formed from a single material by an injection molding process.

3. A device as recited in claim 1 wherein the portion of the tongue scraper/cleaner includes the same material selected from the group consisting of polypropylene, polyethylene, polystyrene, and plastic.

4. A device as recited in claim 1 wherein said neck is oriented at an angle of less than 180 degrees with said handle longitudinal axis.

5. A device as recited in claim 1 further comprising a plurality of tongue scraper blades arranged in rows in the head region of the device; wherein each of said tongue scraper blades has a longitudinal axis; and wherein each of said tongue scraper blades is oriented with respect to said handle so that said tongue scraper blade longitudinal axis is generally orthogonal to said handle longitudinal axis.

6. A device as recited in claim 1 further comprising finger grips located on said handle to enhance secure gripping of the handle.

7. A device as recited in claim 1 wherein at least some of said bristles are angled or curved with respect to said handle longitudinal axis.

8. A device as recited in claim 12 wherein at least some of said bristles are of a length greater than about 5 mm and less than about 10 mm.
